(19) United States
(12) Patent Application Publication
(10) Pub. No.: US 2026/0241065 A1
(43) Pub. Date: Aug. 20, 2026

(54) POROUS 3D MATRIX BASED ON POLYELECTROLYTE COMPLEXES AND USES THEREOF

(71) Applicants: CENTRE NATIONAL DE LA RECHERCHE SCIENTIFIQUE, PARIS (FR); ETABLISSEMENT FRANCAIS DU SANG, LA PLAINE SAINT DENIS CEDEX (FR); INSTITUT NATIONAL DE LA SANTE ET DE LA RECHERCHE MEDICALE, PARIS (FR); UNIVERSITE TOULOUSE III - PAUL SABATIER, TOULOUSE (FR); INSTITUT NATIONAL POLYTECHNIQUE DE TOULOUSE, TOULOUSE (FR); CENTRE HOSPITALIER UNIVERSITAIRE DE TOULOUSE, TOULOUSE (FR)

(72) Inventors: AGNÈS COSTE, TOULOUSE (FR); LISE LEFEVRE, TOULOUSE (FR); MAYLIS FARNO, TOULOUSE CEDEX 9 (FR); BRIGITTE SALLERIN, TOULOUSE CEDEX 09 (FR); SOPHIE GIROD FULLANA, TOULOUSE CEDEX 9 (FR); AUDREY VARIN, TOULOUSE (FR); ALI SANOUJ, TOULOUSE (FR)

(21) Appl. No.: 19/164,733

(22) PCT Filed: Mar. 14, 2024
(86) PCT No.: PCT/EP2024/056879

§ 371 Date: Sep. 12, 2025

(30) Foreign Application Priority Data
Mar. 15, 2023 (FR) FR2302374

(51) Int. Cl. A61L 26/00

(52) U.S. Cl. CPC A61L 26/0052; A61L 26/0023; A61L 26/0066; A61L 26/0085

(57) ABSTRACT

The present invention relates to a porous matrix of polyelectrolyte complexes (PEC) based on alginate and chitosan or derivatives thereof, which is particularly suitable for cell therapy and in particular for the healing of soft tissues and tissue repair. The matrix according to the invention can advantageously be used in combination with macrophages in order to promote wound healing. The invention also relates to a hybrid biomaterial comprising such a porous matrix and pro-wound-healing macrophages, and optionally mesenchymal stromal cells, and to a kit comprising this matrix. The present invention also relates to a method for preparing such a matrix, to a method for preparing such a hybrid biomaterial, and to the use of such a hybrid biomaterial in regenerative medicine.

### TECHNICAL FIELD

The present invention relates to a porous 3D matrix obtained on the basis of polyelectrolyte complexes (PEC) formed between anionic and cationic polymers, which is particularly suitable for cell therapy and in particular for the healing of soft tissues and tissue repair. The matrix according to the invention can advantageously be used in combination with macrophages in order to promote wound healing. The invention also relates to a biomaterial comprising such a porous matrix and cells of interest, such as macrophages and/or mesenchymal stromal cells, and to a kit comprising this matrix. The present invention also relates to a method for preparing such a matrix, to a method for preparing such a biomaterial, and to the use of such a hybrid biomaterial in regenerative medicine.

### TECHNOLOGICAL BACKGROUND

Tissue engineering, or regenerative medicine, generally uses therapeutic cells, substances required for tissue development (signaling molecules, growth factors, etc.) and/or implantable biomaterials.

Generally, an implantable biomaterial intended for deep cell seeding must have biocompatability characteristics in order not to generate an excessive inflammatory response once implanted. In contrast, the material should ideally enable the adhesion of the cells, the normal functioning thereof, the migration thereof and the production of new extracellular matrix. The biomaterial should also promote the viability of the therapeutic cells. Furthermore, the biomaterial should have a highly macroporous, and interconnected, structure so as to enable cell colonization and nutrient diffusion. Finally, the material should have mechanical properties associated with those of the target tissue, so as to reproduce a biomimetic environment for the cells it is accommodating.

Therapeutic cells may be used in the case of wound treatment, and particularly chronic wound treatment. It may be particularly beneficial to administer them via an implantable biomaterial, in order to provide a protective 3D environment which ensures the local administration of viable cells to the wound or more generally the damaged tissue or organ. Chronic wounds, and particularly diabetic food wounds, are “stuck” in a low-intensity inflammatory phase that prevents them from moving on the subsequent steps of wound healing and is suggestive of defective inflammation resolution (Miao et al., 2012). The physiopathological microenvironment of chronic wounds is itself associated with functional macrophage dysregulation. Dysregulation and loss of functionality of macrophages contribute greatly to the defective wound healing of chronic wounds, including diabetic foot wounds.

Thus, macrophages have a key role in orchestrating the wound healing process. The following distinction has recently been proposed (Krzyszczyk et al., 2018) in a way which is relevant and suitable for the context of wound healing where macrophages have a key role: on the one hand, there are pro-inflammatory macrophages which are involved in eliminating pathogens by phagocytosis and which secrete pro-inflammatory cytokines, toxic mediators and reactive oxygen species; and on the other hand, there are pro-wound-healing macrophages and pro-resolving macrophages which are involved in tissue repair and remodeling processes.

One of the most widely-held hypotheses for explaining why inflammation persists in chronic wounds is the absence of a change in phenotype (switch) of pro-inflammatory macrophages to a pro-resolving phenotype.

Macrophages are therefore becoming an increasingly attractive therapeutic target in regenerative medicine. Indeed, in light of the dysfunction of macrophages in chronic wounds and the harmful impact of the lack of a transition from a pro-inflammatory phenotype to a pro-resolving phenotype, it appears to be essential to re-establish the pro-resolving and pro-repairing functionality of macrophages.

Research has focused on developing strategies for controlling inflammation in chronic wounds by acting more or less directly on the phenotype and activity of macrophages. In particular, several complementary approaches have been proposed, such as modulating the phenotype of endogenous macrophages (by promoting a pro-resolving phenotype or by attenuating the pro-inflammatory phenotype), or directly by the local provision of pro-resolving macrophages to the wound.

However, to date, none of the therapeutic wound healing strategies developed involving macrophages has enabled the wounds to satisfactorily return to anatomical and functional integrity.

### SUMMARY OF THE INVENTION

The inventors have researched wound healing and in particular chronic wound healing in diabetics, and have developed a PEC matrix based on anionic and cationic polymers that is particularly suitable for soft tissue wound healing and tissue repair. The matrix according to the invention has mechanical and structural properties such that it can simultaneously serve as a 3D support for cells, and in particular macrophages, and as a wound dressing. The matrix according to the invention is particularly suitable for use in combination with pro-resolving macrophages. In particular, the inventors have developed an alginate/chitosan PEC matrix which can be used as a cell therapy support which is suitable for soft tissues.

The invention therefore relates to a three-dimensional biomaterial comprising a matrix of polyelectrolyte complexes (PEC) based on modified or unmodified alginate and chitosan, and macrophages, said matrix having an open, interconnected macroporosity in which the alginate/chitosan weight ratio is between 20/80 and 80/20.

In one embodiment, the PEC matrix is based on alginate and chitosan, with the alginate/chitosan weight ratio being 40/60.

The alginate can in particular have an M/G ratio of between 1.4 and 2.7, preferentially of equal to 2+/−0.2, a molecular weight (Mw) of between 150 000 and 250 000, and a polydispersity index (PI) of less than 2, preferentially of approximately 1.5.

The chitosan advantageously has a degree of deacetylation (DDA) of between 75% and 90%, preferentially between 75% and 85%, an Mw of between 130 000 and 400 000, preferentially of between 145 000 and 400 000, more preferentially of between 200 000 and 400 000, and a polydispersity index of less than 2, preferentially of approximately 1.7, more preferentially approximately 1.8.

According to the invention, the biomaterial can be a hybrid biomaterial comprising pro-wound-healing macrophages. The pro-wound-healing macrophages are for example pro-resolving macrophages.

In one embodiment, the hybrid biomaterial comprises pro-wound-healing macrophages and mesenchymal stromal cells (MSCs), the pro-wound-healing macrophages/mesenchymal stromal cells ratio preferentially being between 1/99 and 99/1. For example, the pro-wound-healing macrophages/mesenchymal stromal cells ratio is 50/50.

The invention also relates to a method for preparing a hybrid biomaterial according to the invention, comprising the steps of:
(a) culturing monocytes and/or macrophages under conditions that make it possible to obtain pro-wound-healing macrophages;
(b) preparing a matrix of polyelectrolyte complexes (PEC) based on alginate and chitosan in which the two polymers are at a relative proportion of between 40/60 and 60/40;
(c) optionally drying the PEC matrix;
(d) sterilizing the PEC matrix;
(e) seeding the PEC matrix with pro-wound-healing macrophages obtained in step (a), optionally combined with MSCs.

In one embodiment, sterilization step (d) is a sterilization by low-energy pulsed electron beam irradiation or a sterilization by low-energy continuous electron beam irradiation.

As indicated above, in one embodiment, the matrix from step (b) undergoes a drying step before the sterilization step (d).

The invention also relates to a cutaneous wound dressing intended for regenerative medicine, comprising a three-dimensional biomaterial according to the invention.

The invention also relates to pro-wound-healing macrophages, optionally combined with MSCs, for use thereof in regenerative medicine in a subject, characterized in that said pro-wound-healing macrophages and the optional MSCs are in a form suitable for their administration to said subject by means of a three-dimensional biomaterial according to the invention.

Such pro-wound-healing macrophages, optionally combined with MSCs, can be used in particular for the treatment of a wound, in particular a chronic wound.

For example, the subject has diabetes and/or is an elderly subject.

In one embodiment, the three-dimensional biomaterial is in the form of a wound dressing, a patch, or an implantable matrix.

Said pro-wound-healing macrophages and the optional MSCs may have been obtained beforehand from cells from said subject selected from bone marrow cells, iPS cells or blood monocytes, and adipose tissue (ASC: “Adipose tissue-derived mesenchymal Stroma Cell”).

The invention also relates to a kit intended for regenerative medicine, and in particular for the treatment of a wound, such as a chronic wound, said kit comprising:
an alginate and chitosan PEC matrix, said matrix having an open, interconnected macroporosity in which the anionic polymer/cationic polymer weight ratio is between 20/80 and 80/20, preferentially between 40/60 and 60/40;
a culture medium suitable for culturing monocytes or pro-wound-healing macrophages, and/or a culture medium suitable for differentiating bone marrow cells into macrophages of a pro-wound-healing phenotype.

Advantageously, the PEC matrix is sterilized.

### DESCRIPTION OF THE DRAWINGS

FIG. 1 shows the chemical structure of sodium alginate and the monomer units M and G thereof (A), and an example of an alginate chain sequence (B).

FIG. 2 shows the chemical structure of chitin or chitosan. When R=—COCH₃ and x>50%, this is chitin; and when R═H and y>60-70%, this is chitosan.

FIG. 3A shows the distribution of the various groups studied in an in vivo mouse model of the cell therapy strategy for chronic wounds.

FIG. 3B shows the timeline for the test of FIG. 3A, and the experimental conditions.

FIG. 4 shows the gene expression profile for macrophage (mΦ) M0, M1 and M2 after 24 hours of culture or in contact with 40/60 PEC matrices. (A) anti- and pro-inflammatory cytokines. (B) membrane receptors. The analyses were performed in triplicate for each condition (one-way ANOVA test; * P≤0.05; ** P<0.01; *** P≤0.001; **** P≤0.0001). For the sake of clarity on the graphs, only significant differences between the mo in contact with the matrices compared to their reference group (mΦ in culture) are indicated.

FIG. 5AB shows the results of the flow cytometry study of expression of the surface markers F4/80, CD206 (MR) and CD86 by mΦ M2 after 24 hours of culture (A) or in contact with 40/60 PEC matrices (B).

FIG. 5C is a histogram showing the corresponding percentages of the various populations F480+CD206+ and F480+CD86+ from FIGS. 5AB (n=3 replicas).

FIG. 6 is a diagram showing the urea concentrations for 100 000 mΦ M0, mΦ M1 or mΦ M2 after 24 hours of culture or in contact with 40/60 PEC matrices (mΦ: macrophage). The analyses were performed in triplicate for each condition (n=3; one-way ANOVA test; * P≤0.05; ** P≤0.01.; **** P≤0.0001)

FIG. 7 is a graph showing the monitoring, by luminescence, of the production of reactive oxygen species by the macrophages (mΦ) M0, M1 and M2 after 24 hours of culture or in contact with the 3D matrices after stimulation with 100 mM of TPA.

FIG. 8 shows confocal micrographs of a 40/60 PEC matrix after 24 hours of culture and after differential labeling of the living/dead cells. The inset at the top left corner indicates the imaged faces of the matrix. The 3D matrix in its entirety is presented in (A): this is a 3D reconstruction at 10× magnification (10×) with phase contrast and z-projection of the slices. Images B-C-D are the z-projection of a cross section of the matrix at 10×. Images E-F-G and H-I-J are z-projections of the matrix imaged longitudinally, at 10× and 40×, respectively.

FIG. 9 shows the wound closure kinetics from the first day of treatment (D2). The statistical analysis between the 3D matrix+mΦ M2 group and the control group HFD is indicated by * (p<0.05).

FIG. 10AB shows images representing histological sections from the control group (A) and from the group treated with the 3D matrices+mΦM2 (B).

FIG. 10C shows the histological scores associated with the images in FIGS. 10A and 10B.

FIG. 11 shows the viability of different cell types (human M2-Mφ and human ASCs) in the matrix after 2 days and 6 days of culture after labeling using the “ReadyProbes™ Cell Viability Imaging Kit” and analysis of fluorescence using confocal microscopy.

FIG. 12 shows the functionality of the human Mφ and ASCs seeded in the matrix in the basal state or after infectious stimuli (LPS). After 24 h of culture, the concentrations of IL-10, IL-6 and IL-1β are determined using ELISA.

FIG. 13 shows the effects of a matrix according to the invention, seeded with different therapeutic murine cells, on the repair of chronic wounds. A 1 cm² wound is made in the backs of male diabetic C57BL/6 mice which are more than 10 months old. The matrices alone (A), or matrices containing pro-resolving macrophages (M2-Mφ)+combined with adipose tissue mesenchymal stromal cells (ASCs) (B), are applied to the wound. The matrices are placed on the wound for 24 h, then replaced with a new matrix for 3 days. A photo of the wound is taken every day, and the animals are sacrificed when a group reaches 90% wound closure. At a different time, the surface of the wound is measured and the percentage closure is determined relative to the surface area of the wound on D0. (C) Area under the curve after the different treatments.

FIG. 14 shows a comparison of the number of days required to close the wound by 25, 50, 75, 95 and 100% between the untreated mice and the mice treated with the therapeutic cells contained in the matrix. The number indicated on the histograms is the difference in days required to close the wound by X % between the untreated mice (black bars) and the mice treated with the matrix alone (A), the matrix containing M2-Mφ (B), or the matrix containing M2-Mφ combined with ASCs (C).

FIG. 15 shows a histological analysis of the wounds after treatment with the matrix combined with different therapeutic cells. 10 days post-lesion, the animals are sacrificed, the dorsal skin is removed and a histological analysis (hemalum eosin staining) is performed. In order to evaluate wound closure, the distance between the paniculus carnosus on either side of the wound is measured on the histological sections from the mice treated with the matrix alone (A) or with the matrix containing M2-Mφ and ASCs (B). (C) macroscopic analysis of the histological sections (hemalum eosin staining) of the wounds after treatment (10 days post-lesion).

FIG. 16 shows the effects of matrices containing different human pro-resolving cells on the proliferation of human skin fibroblasts. M2-Mφ (A) or M2-Mφ combined with ASCs (M2Mφ +ASCs) (B) are cultured on culture plates (2D) (dashed black line) or on the matrix (3D) (solid black line) for 24 h in α-MEM+0.1% fetal calf serum (FCS). After 24 h of culture, the supernatants are recovered and added to human primary fibroblasts cultured at low density. The proliferation of the fibroblasts cultured in these culture supernatants is evaluated for 7 days using a real-time imaging system (Incucyte S3, Sartorius). A photograph of each well is taken every 6 hours and the rate of proliferation is analyzed by comparing the percentage of the area occupied by the cells in each image at different times. Negative control: 0.1% FCS (light gray line, round). Positive control: 10% FCS (dark gray line, square)

FIG. 17 shows that the matrix promotes the pro-inflammatory profile of the macrophages. The human macrophages are differentiated into anti-inflammatory macrophages (M2-Mφ) (IL-4 for 24 h). The M2-Mφ are then cultured on a plate (2D) or on the matrix (3D). After 24 h, the culture supernatants are recovered and the IL-10 concentration is evaluated using ELISA.

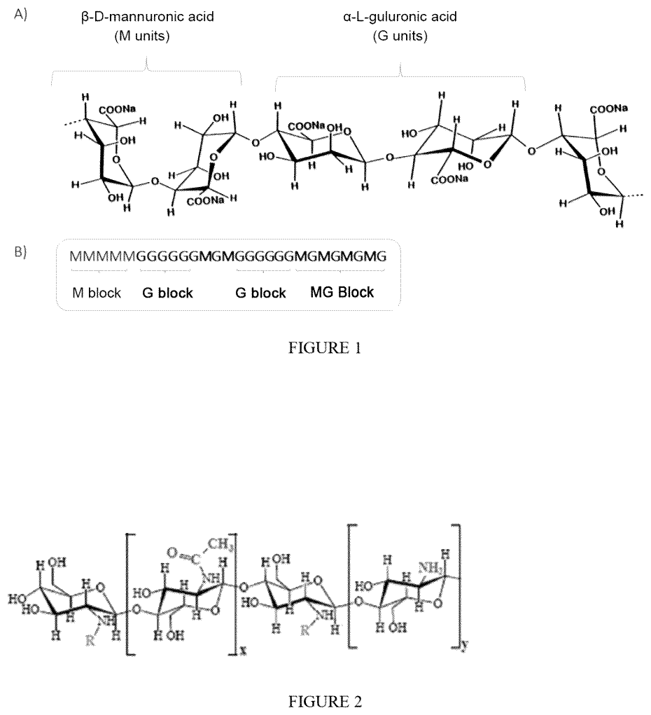

### DETAILED DESCRIPTION OF THE INVENTION

The present invention relates to a matrix of polyelectrolyte complexes that is particularly suitable for use in a wound dressing in the context of tissue regeneration. In particular, the matrix can be used as a biomaterial in combination with therapeutic cells such as macrophages and/or mesenchymal stromal cells (MSCs) to assist in the healing of a wound.

### Definitions

The following definitions apply in the context of the present application.

“Matrix” means a three-dimensional polymeric structure which can be worked to assume the desired form, depending on the final intended use thereof. In the context of the invention, a matrix is advantageously biocompatible and/or biodegradable.

“Biocompatible” means a material, a matrix, or a substance which does not interfere with, and does not degrade, the medium in which it is used.

“Biodegradable” means a material or a matrix which can be decomposed naturally by living organisms. In the case of an implantable material or matrix, this then means a material or a matrix which can be decomposed naturally by the organism in which it is to be implanted.

“Biomaterial” means a non-living material which is intended to interact with a biological system, with an appropriate host response within a specific application. In the context of the invention, a “hybrid biomaterial” is the combination of a matrix and therapeutic cells which can be used in particular as a wound dressing or a temporary skin substitute and which is able to deliver therapeutic substances (bioactive materials secreted by the cells, active principles) and/or to stimulate a cell target.

“Chronic wounds” are wounds which are difficult to heal or which are not following the usual physiological process. A wound is generally considered to be chronic if it has not succeeded in re-establishing anatomical and functional integrity after three months. Chronic wounds generally cover vascular ulcers (venous and arterial), pressure sores, and diabetic feet.

The term “approximately”, associated with a numerical value, means the stated value +/−10%, preferentially +/−5%.

Unless indicated otherwise, when ranges are given, the limit values are included within said ranges.

### Matrix

The present invention is chiefly based on a biocompatible, and advantageously biodegradable, polymer matrix. More specifically, the matrix is a matrix of polyelectrolyte complexes (PEC) based on anionic polymer(s) and cationic polymer(s). PECs have a number of advantages, such as maintaining the very good biocompatibility of the polymers (in this case biobased polymers) from which they are derived. This is why PECs are already conventionally used in tissue engineering with promising results in this field (Ishihara 2019).

The matrix is a three-dimensional (3D) matrix. More specifically, the matrix according to the invention is a PEC matrix having an open, interconnected macroporosity.

“Open, interconnected macroporosity” means that the matrix more particularly comprises pores having a mean diameter of between 50 and 300 μm, that said pores are in communication with one another, and that it is possible to access said pores from the outer surface of the matrix.

According to the invention, the anionic polymer/cationic polymer weight ratio in the matrix is between 20/80 and 80/20. In particular, said ratio is approximately 20/80, 30/70, 40/60, 50/50, 60/40, 70/30, 80/20. Preferentially, said ratio is between 35/65 and 65/35, more preferentially between 35/65 and 45/55, and in particular approximately 40/60.

The biocompatible anionic polymers are advantageously selected from alginates and modified alginates. Alginates form a family of linear copolymers consisting of a sequence of units of β-D-mannuronic acid (M unit) and α-L-guluronic acid (G unit) interconnected by β-1,4 glycosidic linkages. The rigidity of alginate gels can be varied by varying the M/G ratio. For example, it is possible to use sodium alginate. Modified alginates are alginates in which the underlying structure is functionalized with one or more groups, in particular with one or more peptides. For example, it is possible to use alginate functionalized with RGD tripeptides (L-arginine, glycine, L-aspartic acid) that participate in cell adhesion.

The biocompatible cationic polymers are advantageously selected from chitosan and any chitosan derivative which may be functionalized but which retains an overall positive charge. Chitosan is a polysaccharide consisting of a sequence of N-acetyl-D-glucosamine groups interconnected by β-1,4 linkages. The properties of chitosan vary in particular on the basis of its molecular weight and its degree of deacetylation (DDA).

The matrix according to the invention can in particular be a PEC matrix based on alginate and chitosan. The alginate/chitosan weight ratio is between 20/80 and 80/20. Preferentially, said ratio is between 30/70 and 70/30, more preferentially between 35/65 and 45/55, and in particular approximately 40/60.

The alginate used can in particular have an M/G ratio of between 1.4 and 2.7, in particular between 1.5 and 2.5, for example approximately equal to 1.5, to 1.6, to 1.7, to 1.8, to 1.9, to 2, to 2.1, to 2.2, to 2.3, to 2.4, to 2.5.

Advantageously, the alginate has a molecular weight (Mw) of between 150 000 and 250 000, +/−10%, for example of approximately 150 000, 155 00, 160 000, 165 000, 170 000, 175 000, 180 000, 185 000, 190 000, 195 000, 200 000, 210 000, 220 000, 230 000, 240 000, 250 000.

The alginate can have a polydispersity index (PI) preferentially of less than 2, in particular of between 1.9 and 1, preferentially of approximately 1.9, approximately 1.8, approximately 1.7, approximately 1.6, approximately 1.5, approximately 1.4, approximately 1.3, approximately 1.2, approximately 1.1, approximately 1.

The chitosan used can have a degree of deacetylation (DDA) of between 50% and 90%, in particular between 75% and 90%, preferentially between 75% and 85%.

The molecular weight (Mw) of the chitosan is advantageously between 100 000 and 500 000 +/−10%, preferentially between 130 000 and 400 000, between 140 000 and 350 000, between 150 000 and 300 000, between 175 000 and 270 000, between 200 000 and 250 000, in particular approximately 135 000, approximately 140 000, approximately 145 000, approximately 150 000, approximately 155 000, approximately 160 000, approximately 165 000, approximately 170 000, approximately 175 000, approximately 180 000, approximately 185 000, approximately 190 000, approximately 195 000, approximately 200 000, approximately 210 000, approximately 220 000, approximately 230 000, approximately 240 000, approximately 250 000, approximately 260 000, approximately 270 000, approximately 280 000, approximately 290 000, approximately 300 000, approximately 350 000, approximately 400 000.

The polydispersity index (PI) of the chitosan is preferentially less than 2, in particular of between 1.9 and 1, preferentially of approximately 1.9, approximately 1.8, approximately 1.7, approximately 1.6, approximately 1.5, approximately 1.4, approximately 1.3, approximately 1.2, approximately 1.1, approximately 1.

The PEC matrix preferentially comprises alginate and chitosan according to the above characteristics in an alginate/chitosan weight ratio of between 35/65 and 45/55, preferentially of approximately 40/60.

In the context of the invention, the alginates advantageously have an M/G ratio of between 1.4 and 2.7, preferentially of equal to 2+/−0.2, a molecular weight (Mw) of between 150 000 and 250 000, in particular of approximately 195 000, and a polydispersity index (PI) of less than 2, preferentially of approximately 1.5.

Similarly, in the context of the invention, the chitosan advantageously has a degree of deacetylation (DDA) of between 75% and 90%, preferentially between 75% and 85%, an Mw of between 130 000 and 400 000, preferentially of between 140 000 and 250 000, or between 200 000 and 400 000, and a polydispersity index of less than 2, preferentially of approximately 1.8 or approximately 1.7.

Thus, in one example, the alginate used can have an M/G ratio of 2.1+/−0.1, a molecular weight of approximately 193 100, and a polydispersity index of approximately 1.5. Similarly, the chitosan used can have a DDA of 80%+/−5%, a molecular weight of approximately 216 300, and a PI of approximately 1.8.

In another example, the alginate used can have an M/G ratio of 1.4+/−0.1, a molecular weight of approximately 162 000, and a polydispersity index of approximately 1.7. Similarly, the chitosan used can have a DDA of 88%+/−5%, a molecular weight of approximately 146 700, and a PI of approximately 1.7.

In a particular embodiment, the PEC matrix comprises an alginate/chitosan weight ratio of between 35/65 and 45/55, preferentially of approximately 40/60. The alginate in the matrix has an M/G ratio of 2.1+/−0.1, a molecular weight of approximately 193 100 and a PI of approximately 1.5, and the chitosan has a DDA of 80%+/−5%, a molecular weight of approximately 216 300 and a PI of approximately 1.8.

The matrix according to the invention is suited to the deep seeding and immobilization of therapeutic cells, and has absorbent properties that are particularly suited to use as a wound dressing for the absorption of exudates.

The matrix according to the invention can be prepared by any suitable method known to those skilled in the art. In particular, it is possible to employ the preparation method described in application WO2017/017379. After being produced, the matrix can be sterilized and optionally dried or lyophilized, with this drying or lyophilization preferentially being carried out before the sterilization with a view to the subsequent use of the matrix as a biomaterial.

Advantageously, the matrix of polyelectrolyte complexes (PEC) is produced by mixing the anionic polysaccharide, such as the alginate, with the cationic polymer, such as the chitosan, in proportions of between 40/60 and 60/40. In particular, the cationic polymers and the anionic polymer are in proportions by weight of between 40/60 and 60/40. In particular, the cationic polymers and the anionic polymer are in proportions that enable a maximum degree of ionic interaction (+/−10%).

For example, the step of preparing a PEC matrix based on chitosan and alginate comprises:
mixing an alginate solution and a chitosan solution;
molding and freezing the PEC mixture obtained;
lyophilizing the PEC mixture;
gelling the lyophilized PEC matrix by adding a 0.1 M CaCl2 solution;
rinsing, optionally refreezing, then optionally re-lyophilizing said matrix.

The matrix according to the invention can be dried, for example by drying using supercritical CO₂, and/or be lyophilized, in order to promote preservation and storage thereof before it is used.

The matrix according to the invention can advantageously be sterilized, preferentially under dry conditions. The sterilization can be performed using any physical or chemical means known to those skilled in the art and suitable for PEC matrices. In particular, it is possible to sterilize the matrix using low-energy pulsed electron beam irradiation or low-energy continuous electron beam irradiation. Low-energy irradiation means that having an energy of less than 500 keV. The sterilization method described in Farno et al 2021 (“Low-energy electron beam sterilization of solid alginate and chitosan, and their polyelectrolyte complexes”, Carbohydrate Polymers 261 (2021) 117578) is particularly suitable.

In one embodiment, the sterilization is performed using irradiation at a dose of less than 5 kGy, in particular of between 2 and 3 kGy, preferentially of 2.5 kGy, by pulsed technology with a beam energy of between 200 and 350 keV, preferentially between 250 and 300 keV, in particular at 280 keV.

In another embodiment, the sterilization is performed using irradiation at a dose of between 10 and 25 kGy, preferentially of between 12 and 20 kGy, more preferentially of 15 kGy, by pulsed technology with a beam energy of between 350 and 550 keV, preferentially between 400 and 450 keV. For example, the sterilization is performed using irradiation at a dose of 15 kGy with a beam energy of 430 keV.

Such sterilizations advantageously make it possible to retain the porosity and architecture of the matrix according to the invention.

### Hybrid Biomaterial

The invention also relates to a biomaterial combining a matrix as described above and therapeutic cells and/or therapeutic substances. The matrix according to the invention has mechanical and biological properties which are particularly suited to cell therapy. In particular, the matrix according to the invention has properties which are favorable to the seeding and survival of particular cells, such as macrophages and mesenchymal stromal cells. This is because the porosity generated enables deep seeding of the matrix, and the architecture reproduces a biomimetic environment capable of maintaining cell viability, a particular phenotype and/or metabolic activity.

The matrix according to the invention is thus particular suited to the seeding and survival of macrophages and/or mesenchymal stromal cells (MSCs). It is thus possible to use the matrix according to the invention with macrophages or with a combination of macrophages and MSCs.

It is known that infection of a chronic wound causes significant wound healing delays. Indeed, in 90% of chronic wounds, a biofilm has developed, representing a significant obstacle to wound healing. Thus, moving on to the inflammation resolution phase can only be achieved if this biofilm is eliminated. Pro-inflammatory macrophages, which are involved in eliminating pathogens by phagocytosis and by secreting pro-inflammatory cytokines, toxic mediators and reactive oxygen species, are therefore essential in controlling wound infection.

Furthermore, in the wound healing process, pro-resolving macrophages are essential in halting inflammation and guiding wound healing toward the repair phase. In the final phases of wound healing, the goal of these macrophages is a return to homeostasis and the maturation of the skin to its initial state. Inflammation resolution is partly activated by the phagocytosis of apoptotic PNNs by macrophages, which secrete TGF-β and VEGF as a result. Pro-resolving macrophages also produce large quantities of IL-10, known to be immunosuppressive. They also actively contribute to regulating the extracellular matrix (ECM) and to remodeling it by secreting various proteases such as MMP and their inhibitors, TIMPs. Similarly, these macrophages are involved in the apoptosis of myofibroblasts, so as to minimize the risk of excessive fibrosis. Pro-resolving macrophages are characterized by the presence of PRR (pattern recognition) receptors which are capable of recognizing pathogen-associated molecular patterns (PAMPs) and/or damage-associated molecular patterns (DAMPs).

Thus, the matrix according to the invention advantageously comprises pro-wound-healing macrophages. According to the invention, “pro-wound-healing macrophages” denotes pro-inflammatory macrophages, pro-resolving macrophages, or a mixture thereof.

The addition of pro-inflammatory/anti-bacterial and/or pro-resolving macrophages to the matrix is advantageously conditioned on the infectious and inflammatory status of the wound to be treated. Those skilled in the art are able to decide on the type of pro-wound-healing macrophages to be used with the matrix depending on the wound to be treated.

Particularly surprisingly, the inventors have demonstrated that the matrix according to the invention makes it possible to maintain the macrophages of the matrix in a pro-resolving state.

Advantageously, the matrix according to the invention comprising pro-resolving macrophages can make it possible to force endogenous macrophages in contact with said matrix toward a pro-resolving phenotype. Thus, in the case of the use of the matrix as a wound dressing applied to a wound, said matrix can promote wound healing by guiding the macrophages present in the wound toward a pro-resolving phenotype.

The matrix according to the invention can also be used with other types of therapeutic cells, such as MSCs, and/or with therapeutic molecules, and can optionally be functionalized with antibiotics or natural substances (propolis, proanthocyanidin A). MSCs are particularly beneficial in the context of wound treatment, in combination with macrophages, as they make it possible to induce a pro-resolving phenotype in the macrophages. The polarization of the macrophages toward a pro-resolving phenotype by means of MSCs can involve both exogenous macrophages provided by the biomaterial and endogenous macrophages present in the wound to which the hybrid biomaterial is applied. Furthermore, by secreting paracrine factors, MSCs can modulate and recruit cells endogenous to the wound, such as fibroblasts and keratinocytes, and thereby accelerate wound closure (Shingyochi, Y, 2015, Exp Opinion mol Ther; Krzyszczyk, P, 2018, Front Physiol). MSCs also serve a central role in wound neovascularization, by secreting anti-inflammatory factors and also by stimulating the production of VEGF by the cells in the microenvironment (Shingyochi, Y, 2015, Exp Opinion mol Ther). MSCs thus have a significant potential to accelerate the wound closure process due to their anti-inflammatory properties, by stimulating angiogenesis and by producing soluble factors that improve the wound microenvironment, and thus promote tissue reconstruction. Thus, the MSCs of the matrix may act in the different phases of the healing of healthy or infected wounds.

The MSCs used may for example originate from adipose tissue (ASC), bone marrow, umbilical cord, or be IPS cell derivatives.

Interestingly, the inventors have demonstrated that the combination of pro-resolving macrophages and ASCs significantly increases the number of viable cells in the matrix.

Advantageously, the combination of pro-resolving macrophages and ASCs potentiates the pro-resolving and therapeutic activity of the cells seeded in the matrix.

In a particular embodiment, the biomaterial according to the invention comprises a matrix based on alginate and chitosan, advantageously having an alginate/chitosan weight ratio of approximately 40/60, and macrophages, preferentially pro-wound-healing macrophages, in particular pro-resolving macrophages.

In a particular embodiment, the hybrid biomaterial according to the invention comprises a matrix based on alginate and chitosan, advantageously having an alginate/chitosan weight ratio of approximately 40/60, pro-wound-healing macrophages and mesenchymal stromal cells.

In a particular embodiment, the hybrid biomaterial according to the invention comprises a matrix based on alginate and chitosan, advantageously having an alginate/chitosan weight ratio of approximately 40/60, pro-wound-healing macrophages, preferentially pro-resolving macrophages, and mesenchymal stromal cells.

In one embodiment, the hybrid biomaterial according to the invention consists of a matrix as described above and pro-wound-healing macrophages, preferentially pro-resolving macrophages. Alternatively, the hybrid biomaterial according to the invention consists of a matrix as described above, pro-wound-healing macrophages and MSCs.

When the hybrid biomaterial according to the invention contains macrophages, the concentration by volume of cells in the PEC matrix is between 800 and 8000 cells/mm3 of PEC matrix, preferentially between 500 cells and 4000 cells/mm3 of PEC matrix.

The macrophages having a pro-wound-healing phenotype, and in particular a pro-resolving phenotype, preferentially represent at least 60% by number of the macrophages contained in the PEC matrix, more preferentially at least 70%, 80%, 85%, 90%, 95%.

When the hybrid biomaterial according to the invention contains mesenchymal stromal cells, the concentration by volume of MSCs in the PEC matrix is preferentially between 800 and 8000 MSC/mm3 of PEC matrix.

When the biomaterial according to the invention contains macrophages and MSCs, the macrophages/mesenchymal cells ratio is advantageously between 1/99 and 99/1. For example, the macrophages/mesenchymal cells ratio can be 50/50.

The hybrid biomaterial according to the invention can be prepared by any method known to those skilled in the art.

In general, the method for preparing a hybrid biomaterial according to the invention can comprise steps of preparing a matrix of polyelectrolyte complexes (PEC) according to the invention, optionally drying, sterilizing said PEC matrix, and seeding the PEC matrix with the therapeutic cells, in particular the pro-wound-healing macrophages.

In particular, once the matrix as described above has been produced, dried and sterilized, said matrix is seeded with therapeutic cells by bringing the matrix into contact with a culture medium comprising said therapeutic cells of interest. Of course, it is possible to directly seed the matrix according to the invention without conducting a drying step.

Thus, the method for preparing a hybrid biomaterial according to the invention can comprise the steps of:
(a) culturing monocytes and/or macrophages under conditions that make it possible to obtain pro-wound-healing macrophages;
(b) preparing a matrix of alginate-chitosan polyelectrolyte complexes (PEC) in which the two polymers are in relative proportions of between 40/60 and 60/40;
(c) optionally drying the PEC matrix;
(d) advantageously sterilizing the PEC matrix;
(e) seeding the PEC matrix with pro-wound-healing macrophages obtained in step (a), optionally combined with MSCs.

Depending on the intended use, and in particular on the healing stage of the wound to be treated, step (a) can comprise culturing macrophages under conditions that make it possible to obtain macrophages of the pro-resolving phenotype. For example, step (a) of culturing macrophages comprises culturing bone marrow cells which have previously been taken from a human or non-human mammal in a culture medium that makes it possible to obtain naive macrophages, and polarizing toward a pro-resolving phenotype by culturing them in a culture medium supplemented with IL-4, IL-13 and dexamethasone for several days, in particular between 2 and 10 days, for example at least 4 days. The macrophages are polarized toward a pro-inflammatory phenotype by culturing them in a culture medium supplemented with interferon-γ and/or LPS for several days, in particular between 2 and 10 days, for example at least 4 days.

Alternatively, it is possible to culture iPSs, blood monocytes, etc., in a culture medium suitable for differentiating said cells into macrophages, and to polarize them toward a pro-resolving phenotype (Douthwaite H. et al. 2022 Bio Protoc.). Those skilled in the art are able to adapt this cell preparation step on the basis of the origin of the cells and on the final intended use.

If the matrix is to be seeded with pro-inflammatory macrophages, step (a) can comprise culturing macrophages under conditions that make it possible to obtain macrophages of the pro-inflammatory phenotype. For example, step (a) of culturing macrophages comprises a step of differentiation in the presence of MSCF and a step of culturing for 24 h in the presence of pro-inflammatory cytokines, such as interferon-gamma, and/or of bacterial cell walls, in a defined medium.

In the case in which the cells to be seeded are MSCs, step (a) can comprise isolating cells from the adipose tissue, bone marrow or umbilical cord of a human or non-human mammal, and culturing them in a suitable culture medium. The MSCs are obtained after a step of digesting the tissue. The cells obtained are cultured in MEM alpha medium supplemented with human platelet lysate or fetal calf serum. The cells will be used after at least one culturing.

Of course, it is possible to perform several steps (a) in order to seed several cell types in the matrix.

Step (b) of preparing the PEC matrix and step (d) of sterilization can be carried out as described above.

Of course, the cell culture step (a) and steps (b) and (d) can be carried out independently and at different moments in time. For example, it is possible to prepare the matrix and sterilize it several hours, days, months, etc., before the cell culture step. Before seeding, the matrices can be conserved in the lyophilized state and sterilized in sterilization packaging.

Advantageously, step (e) of seeding comprises bringing the lyophilized and sterilized PEC matrix into contact with a cell pellet (for example a cell pellet of macrophages and/or of mesenchymal cells) then centrifuging said matrix before adding complete medium.

The seeded matrices can be conserved in culture. For example, the seeded matrices are placed in multi-well cell culture dishes to which a volume of medium suitable for the size of the wells and the matrices is added, so as to submerge the seeded matrix. The dishes are conserved in an incubator at approximately 37° C. and 5% CO₂. The conservation duration can depend on the cell type. The culture medium can be refreshed at a frequency which depends on the cell type. Those skilled in the art know how to adapt the culture medium, the volumes and the refreshing of the medium to the cell types and to the final intended use of the biomaterial.

In the case of a matrix seeded with pro-resolving macrophages, the conserving in culture can extend over a period of several days, and in particular 10 days, 15 days, 20 days, 21 days or more, with a very high level of viability of the macrophages.

It is also possible to freeze the matrix after seeding.

### Kit

The invention also relates to a kit intended for regenerative medicine, and in particular for the treatment of a wound, such as a chronic wound, said kit comprising:
a PEC matrix based on alginate and chitosan or derivatives thereof, as described above;
a culture medium suitable for the cells intended to seed the matrix.

For example, the culture medium is suited to the culture of pro-resolving macrophages and/or MSCs.

For example, the macrophages are cultured in supplemented RPMI 1640 and the MSCs are cultured in supplemented alpha-MEM.

The kit can also comprise a culture medium suitable for the differentiation of cells such as bone marrow cells, iPSs, etc. into macrophages of a pro-resolving and/or pro-inflammatory phenotype.

Advantageously, the PEC matrix is a matrix based on alginate and chitosan, as described above.

The PEC matrix is advantageously sterilized and optionally packaged in sterilization packaging, in which it can be conserved until it is used for cell seeding.

The kit according to the invention can also comprise a matrix as described above, already seeded with pro-wound-healing macrophages and optionally MSCs. Such a seeded matrix can be provided in frozen form. The kit can then comprise a medium suitable for the thawing of the matrix and the survival of the therapeutic cells.

According to the invention, the kit can comprise a matrix as described above, shaped to be used as a wound dressing, or provided with means enabling use as a wound dressing. Thus, the kit can contain a tape or any adhesive in order to enable the matrix to be held in position on a wound. In another embodiment, the matrix can be provided in a format that enables it to be cut up in order to adapt it to the size/shape of the wound.

### Uses

The matrix according to the invention can be used alone or in the form of a biomaterial in combination with pro-wound-healing macrophages and/or therapeutic molecules.

The matrix according to the invention can in particular be used as a wound dressing to be applied to a wound. Indeed, as indicated above, such a matrix is able to promote the polarization of endogenous macrophages present in the wound to be treated, for example, toward a pro-resolving phenotype. Thus, the application of a wound dressing comprising such a matrix can aid in the healing of a wound.

Similarly, it is possible to use the matrix according to the invention in combination with one or more therapeutic molecules and/or biological substances. For example, it is possible to load the matrix with antibiotics or natural substances which are intended to reduce the risk of a wound to which said matrix is intended to be applied becoming infected. It is also possible to load the matrix with molecules which are able to promote a cell phenotype, and in particular to promote the pro-resolving phenotype for macrophages (for example, IL-13, IL-4, dexamethasone, eicosanoids). Thus, when the matrix is applied to a wound, said molecules will assist wound healing by promoting the polarization of endogenous macrophages toward a pro-resolving phenotype.

The matrix according to the invention can advantageously be used in combination with macrophages, and more particularly pro-resolving and/or pro-inflammatory macrophages, and/or MSCs, in regenerative medicine and more particularly in the context of treating chronic wounds.

Regenerative medicine means the repair, replacement or regeneration of damaged cells or tissues. According to the invention, tissue repair and the healing of wounds to the skin are mainly targeted.

Thus, the invention relates to pro-wound-healing macrophages for use thereof in regenerative medicine in a subject, said pro-wound-healing macrophages being in a form suitable for their administration to said subject by means of a hybrid biomaterial according to the invention. The invention relates in particular to pro-resolving macrophages, optionally in combination with MSCs, for use thereof in regenerative medicine in a subject, said pro-resolving macrophages being in a form suitable for their administration to said subject by means of a hybrid biomaterial according to the invention.

A subject means a human or non-human mammal in need of medical treatment, such as the treatment of a wound, in particular a chronic wound. Preferentially, the subject is a child, adolescent or adult human mammal. Preferentially, the subject has a disease which limits wound-healing mechanisms, and/or is an elderly subject. In particular, the subject has diabetes, preferentially type II diabetes. Elderly subject means a human mammal aged 70 years or more.

The invention particularly targets pro-resolving macrophages, optionally in combination with MSCs, in a hybrid biomaterial according to the invention, for the use thereof for the treatment of a wound, in particular a chronic wound.

The invention also targets pro-inflammatory macrophages, optionally in combination with MSCs, in a hybrid biomaterial according to the invention, for the use thereof for the treatment of a wound, in particular a chronic wound.

Such a use is particularly suitable for subjects that have a disease which limits wound healing mechanisms, such as diabetes.

Said pro-wound-healing macrophages and/or MSCs can be obtained from cells from said subject, such as blood monocytes, MSCs from adipose tissue, bone marrow or umbilical cord, macrophages, bone marrow cells and/or iPSs.

Advantageously, the three-dimensional biomaterial is in the form of a wound dressing, a patch, or an implantable matrix.

The invention also targets the use of a biomaterial according to the invention, in particular in the form of a wound dressing or implantable matrix, in regenerative medicine. In particular, the invention targets the use of such a biomaterial, according to which said biomaterial is brought into contact with damaged tissue such as a skin wound.

The invention also targets a method for treating damaged tissue, according to which a biomaterial as described above is applied to the damaged tissue in order to promote the repair of said tissue. In particular, the invention targets a method for treating a wound, and particularly a chronic wound, according to which said biomaterial is applied to the wound in order to promote wound healing. In such a case, the hybrid biomaterial advantageously contains pro-wound-healing macrophages and in particular pro-resolving macrophages, optionally in combination with MSCs. The biomaterial can be applied in the form of a wound dressing which is able to be held on the wound.

The invention also targets the use of macrophages for preparing a biomaterial according to the invention for the treatment of damaged tissue. In particular, the invention targets the use of macrophages for preparing a biomaterial according to the invention for the treatment of a wound, more particularly of a chronic wound, such as a chronic diabetic wound.

### EXAMPLES

### Materials and Methods

### A) Matrix

The polymers used are a “medium viscosity” alginate sold by Sigma (reference A-2033; batch 051M0054V) or Algaia (Satialgine XPS 170NS) and a “medium molecular weight” chitosan, also sold by Sigma (reference 448877; batch STBF8484V or batch BCCJ1419).

For the alginate from Sigma, an M/G ratio of 2.1 was determined by the method described by Vilén et al. (Vilén et al., 2011). The weight-average (Mw) and number-average (Mn) molecular weights of the alginate were determined by size exclusion chromatography, and are 193 100 and 126 900 g/mol, respectively, with a polydispersity index of 1.5.

For the chitosan, the degree of deacetylation (DDA) was calculated by applying the method of Heux et al. (Heux et al., 2000) and was estimated to be 80%+/−5%. The Mw and Mn values obtained by size exclusion chromatography are 216 300 and 120 300 g/mol, respectively, with a polydispersity index of 1.8, for the alginate of batch STBF8484V. For the second alginate, the Mw and Mn values obtained by size exclusion chromatography are 146 700 and 86 500 g/mol, respectively, with a polydispersity index of 1.7.

The PEC matrices are obtained by mixing an alginate solution having a fixed concentration of 3% (weight/volume) and a chitosan solution with 1.5% acetic acid and a variable chitosan concentration depending on the desired final polymer ratio in the matrices.

In brief, the alginate and chitosan solutions are homogenized by mechanical stirring and are then mixed in equivalent proportions by weight. After the two polyelectrolytes have been added, the mixture is also mechanically stirred. The final PEC mixture is then placed in a culture plate, frozen for at least 24 hours at −20° C., then the matrices are lyophilized for 24 hours. The PEC matrices are gelled for 1 hour with a 0.1 M calcium chloride solution. The matrices are then rinsed, frozen and lyophilized again.

This protocol makes it possible to obtain a family of 3D matrices having alginate/chitosan ratios ranging from 80/20 to 20/80.

### B) Cells

The murine macrophages used originate from primary culture. They are extracted from the bone marrow of C57BL6/JRj mice (Janvier Labs). The murine ASCs are obtained by enzymatic and mechanical digestion of inguinal adipose tissue from C57BL6/JRj mice (Janvier Labs).

After euthanasia of the mice using carbon dioxide overdose, the femurs and tibias are removed and cleaned so as to remove the surrounding muscles. After the bones have been disinfected in 70% ethanol, the two epiphyses from each bone are removed, then the medullary cavity is washed by injecting PBS in order to recover the cells from the bone marrow. The cell suspension is mechanically dissociated using a pipette by successively taking up and re-ejecting the suspension, then the suspension is filtered at 40 μm and centrifuged for 10 minutes at 1500 rpm. The cell pellet is then taken up in 1 mL of ACK buffer in order to lyse the red blood cells. The pellet is taken up in 9 mL of complete medium (DMEM glutamax, 10% FCS, 1% penicillin/streptomycin and 1% L-glutamine) and centrifuged for a second time. The final pellet is taken up in 5 mL of complete medium; 10 μL are taken off for staining with trypan blue and counting using a Malassez chamber. An average of between 20 and 40 million cells are recovered from the bone marrow per mouse. All the cells are taken up in complete medium supplemented with M-CSF (30 ng/mL); they are seeded in a petri dish to give 300 000 cells/cm². After 4 days of adhesion, macrophages M0 (mΦ M0) are obtained (Martinez et al., 2006; Xia Zhang et al., 2008).

Four days after their isolation and differentiation, the macrophages M0 are not activated and can be polarized toward a pro-resolving phenotype M2 (m\tilde{y} M2) with complete medium supplemented with M-CSF (30 ng/mL), IL-4 (10 ng/ml) and dexamethasone (10-7M) for 3 days, or toward a pro-inflammatory phenotype M1 (mΦ M1) with complete medium supplemented with M-CSF (30 ng/ml), IFN-γ (2 ng/ml) and LPS (100 ng/ml) for 24 hours.

The human ASCs are isolated from human adipose tissue obtained after abdominal dermolipectomy. The adipose tissue is digested by mechanical action, then enzymatic action. The cell pellet represents the stromal vascular fraction of the adipose tissue, which contains the ASCs. The SVF is then cultured, and the ASCs are obtained after 8 days of culture.

The human monocytes are isolated from peripheral blood mononuclear cells (PBMCs) obtained from leukocyte concentrates obtained from different blood donors. The PBMCs are isolated by Ficoll gradient centrifugation. The PBMC layer is collected and the monocytes are isolated by adherence or following magnetic separation. After one week of culture, the macrophages obtained are stimulated for 24 hours, either with 100 ng/mL of M-CSF to obtain non-polarized macrophages (M0-Mφ), or 20 ng/ml of IL-4 to induce a pro-resolving phenotype (M2-Mφ).

### C) Seeding

To recover the macrophages, the culture medium is removed and replaced with cold PBS containing 2 mM EDTA, a calcium chelator. After 5 minutes on ice, the macrophages are gently detached using a cell scraper. The suspension is centrifuged for 5 minutes at 1500 rpm before being stained with trypan blue for counting using a Malassez chamber.

In the experiments for contacting the 3D matrices, the non-activated macrophages M0 or polarized macrophages M1 (pro-inflammatory phenotype) or M2 (pro-resolving phenotype) are cultured for two hours. After adhesion of the macrophages, the matrices are placed in the well so that they are in direct contact with the cells. The cells or the culture supernatant are recovered 24 hours later for the various analyses.

For the in vitro test, the matrices in the lyophilized state are seeding by depositing a 15 μL cell pellet containing 400 000 macrophages. The matrix rapidly absorbs the cell pellet, and is then centrifuged for 1 minute at 400 g in order to facilitate the penetration of the cells throughout the depth of the 3D matrices. Finally, complete medium is added gently at the edge of the well, and the matrices are incubated (37° C., 5% CO2).

D) Recovery of Macrophages after Seeding in the 3D Matrices

The first step consists in cutting the matrices into 4 in order to maximize contact between the cells attached to the matrix and the detachment solution.

The samples are then incubated with a detachment solution before filtration. The debris are filtered successively over a 100 μm screen then a 40 μm screen, then the cell suspensions are centrifuged.

### E) Study of Gene Expression by RT-PCR

Reverse Transcription Polymerase Chain Reaction (RT-PCR) takes place in three successive steps: the extraction of messenger RNA from the cells, the reverse transcription of the mRNA to give cDNA, then the reaction for the amplification of the cDNA by PCR. The macrophages seeded in the 3D matrices were detached beforehand. The pellet of detached macrophages, and the macrophages cultured in wells (“2D” controls), are frozen at −80° C. in 100 μl of lysis buffer (Promega).

mRNA Extraction

The kit “ReliaPrep™ RNA Cell Miniprep System” (Promega) is used to extract the mRNA, following the protocol provided by the supplier. The mRNAs are then assayed using the Nanodrop® (ND-1000).

### Reverse Transcription

The reverse transcription of the mRNAs to cDNA is performed using the “Superscript Vilo cDNA synthesis kit” (Invitrogen). For each experiment, a pool of pure cDNA is formed by mixing 2 μL of each sample. The pool is then diluted to 1/5, 1/10, 1/20 and 1/40 to form a standard curve. The remaining cDNAs are then diluted to ⅕ in water without RNAse/DNAse.

### PCR

The cDNAs are amplified using the “SYBR Green I-based real-time PCR” kit (Roche) in a LightCycler® 480. The buffer sequences used are detailed in the table below. The relative expression of each gene in the samples is determined relative to the standard curve and relative to the housekeeping gene (Gapdh).

TABLE 1

| Gene | Primer | Sequence |
|---|---|---|
| Gapdh | Sense | 5′ AAC-TTT-GGC-ATT-GTG-GAA-GG 3′ |
| | Antisense | 5′ ACA-CAT-TGG-GGG-TAG-GAA-CA 3′ |
| IL-10 | Sense | 5′ TTT-TCA-CAG-GGG-AGA-AAT-CG 3′ |
| | Antisense | 5′ CCA-AGC-CTT-ATC-GGA-AAT-GA 3′ |
| Tgf-β1 | Sense | 5′ AGG-TTG-GCA-TTC-CAC-TTC-AC 3′ |
| | Antisense | 5′ AGG-GGC-CTC-TAA-GAG-CAG-TC 3′ |
| Tnf-α | Sense | 5′ AGG-CTG-TGC-ATT-GCA-CCT-CA 3′ |
| | Antisense | 5′ GGG-ACA-GTG-ACC-TGC-ACT-GT 3′ |
| Mrc1 | Sense | 5′ ATG-CCA-AGT-GGG-AAA-ATC-TG 3′ |
| | Antisense | 5′ TGT-AGC-AGT-GGC-CTG-CAT-AG 3′ |
| Cd36 | Sense | 5′ GCA-GAA-TCA-AGG-GAG-AGC-AC 3′ |
| | Antisense | 5′ GAG-CAA-CTG-GTG-GAT-GGT-TT 3′ |

### F) Study of Surface Markers by Flow Cytometry

In order to study the expression of certain surface markers, the macrophages cultured in wells (2D control) and those detached from the 3D matrices are analyzed by flow cytometry. The cell suspensions are centrifuged, then the cell pellets are incubated in darkness for 20 minutes at 4° C. in Fc block buffer (saturation of non-specific sites). Labeling is then carried out in darkness for 15 minutes at room temperature with the following antibodies diluted in FACS buffer: F4/80+ FITC Biolegend), CD206 APC (Biorad) and CD86 PE (Biolegend). The cell suspensions are then rinsed and centrifuged before being incubated with Live/dead violet (miltenyibiotec). The samples are finally taken up in 186 FACS buffer before acquisition thereof using the BD Fortessa cytometer (BD Biosciences). Analyses are performed by means of the BD FACS DVIA™ software (BD Biosciences).

Fc block buffer: FACS buffer supplemented with 3% mouse serum, 3% rat serum and the anti-CD16/CD32 antibody pair to ½00

FACS buffer: PBS supplemented with 5% FCS and 5 mM EDTA

### G) Quantification of the Production of Reactive Oxygen Species (ROS)

The production of reactive oxygen species (ROS) by the macrophages, seeded or not seeded in 40/60 PEC matrices, was measured by chemiluminescence in the presence of 5-amino-2,3-dihydrophthalazine-1,4-dione (luminol) using a luminometer (Envision, PerkinElmer). The generation of chemiluminescence was monitored continuously for 30 minutes after incubating the cells and/or biopolymeric matrices with luminol (66 μM) then for one hour after stimulation with tetradecanoylphorbol (TPA: 100 nM). Statistical analysis was performed using the area under the curve expressed as number x seconds.

### H) Study of Arginase Activity

The measure of arginase activity is determined from the cell lysates using colorimetric assay of urea production.

### I) In Vivo Experimental Protocol

All the experiments on mice were carried out in compliance with the current regulations regarding the use of animals for scientific purposes.

The mice used in this in vivo test are 8-week-old male C57B1/6NRJ mice (Janvier Labs). All the animals are kept in a daily cycle split into 12 hours of darkness followed by 12 hours of light, at 22° C., with unlimited access to water and food. In order to induce type II diabetes, a high-fat diet (HFD) is given to the mice for 4 months (260 HF diet, Safe) (Gálvez et al., 2019). The mice are weighed at regular intervals and their fasting blood sugar level is measured to verify hyperglycemia.

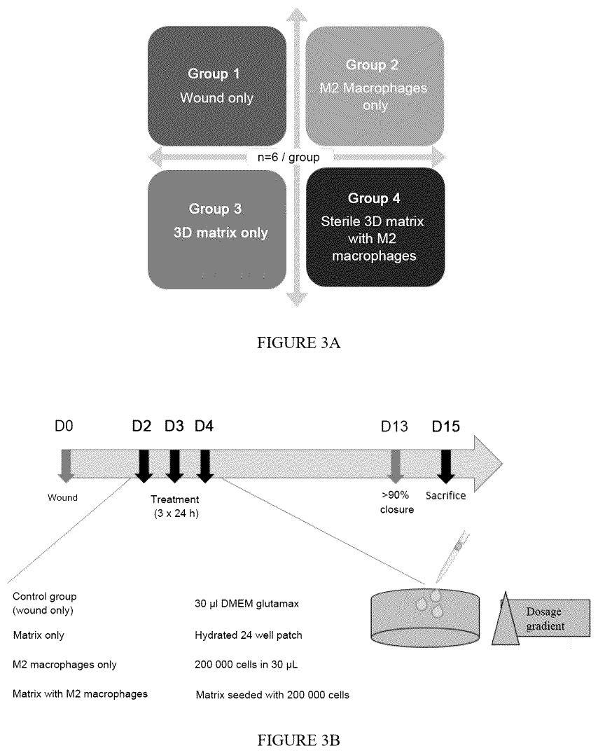

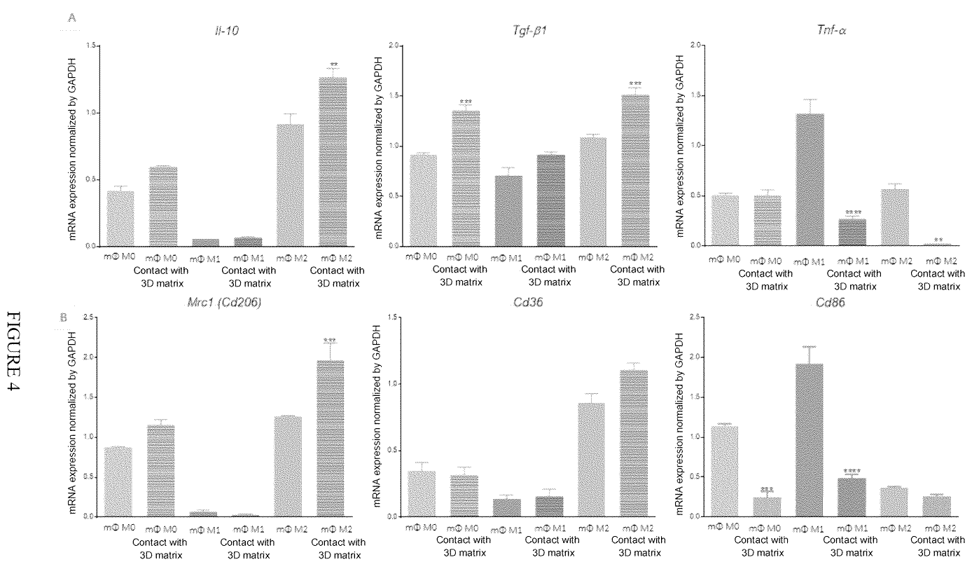

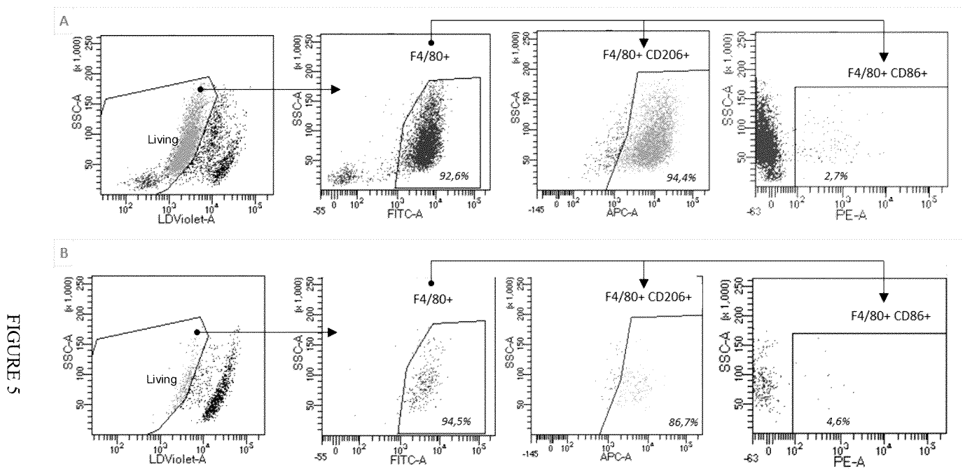

For the first protocol, the mice are distributed randomly into 4 groups of six mice (FIG. 3A). The protocol carried out lasts around ten days (FIG. 3B) and begins by making a wound on day DO. To do this, the mice are anesthetized using isoflurane. The dorsal part and the flanks are shaved and disinfected, then a circular excision of approximately 1 cm² is made. Immediately thereafter, a non-invasive experimental device, developed by PharmaDev (FR2984719/FR2984722), is implanted. This makes it possible, inter alia, to keep the 3D matrices on the wounds.

After the operation, the mice are placed in a heated cage until they awake, and food is placed directly in their cage to facilitate feeding.

After 48 hours (day D2), the first topical treatments are applied:
Control group: 30 μL of culture medium (DMEM glutamax);
“3D matrix only” group: 3D matrix sterilized by pulsed electron irradiation and hydrated in culture medium;
“M2 Macrophages only” group: 200 000 polarized M2 macrophages in vitro;
“3D matrix with M2 macrophages” group: 3D matrix sterilized by pulsed electron irradiation, seeded with polarized M2 macrophages in vitro, and hydrated.

These treatments are left for 24 hours and refreshed twice on days D3 and D4.

On the day on which the mice are sacrificed, skin explants are taken and embedded in paraffin. After sectioning using a microtome (HM ³40^{E} Rotary Microtome, Thermo Scientific™), the sections are stained with hematoxylin and eosin (HE staining). Finally, the slices are scanned for analysis (NanoZoomer Digital Pathology, Hamamatsu).

For the second protocol in vivo, the mice are distributed randomly into 4 groups of five mice. The protocol carried out lasts around ten days and begins by making a wound on day DO (cf in vivo protocol, FIG. 3). After 48 hours (day D2), the first topical treatments are applied:

Control group: 30 μL of culture medium (DMEM glutamax);

“3D matrix only” group: 3D matrix irradiated with UV and hydrated in culture medium;

“3D matrix with M2 macrophages” group: 3D matrix irradiated with UV, seeded with polarized M2 macrophages in vitro, and hydrated.

“3D matrix with M2 macrophages and ASC” group: 3D matrix irradiated with UV, seeded with polarized M2 macrophages in vitro and ASCs, and hydrated.

These treatments are left for 24 hours and refreshed twice on days D3 and D4.

### Viability of Human Macrophages and Human ASCs in the Matrix

Human pro-resolving macrophages (M2-Mφ: 2×10⁵ cells) and human ASCs (2×10⁵ cells) are seeded alone or in combination in the 3D matrix and are cultured. After 2 and 6 days of seeding, the cells present in the matrix are labeled with the “ReadyProbes™ Cell Viability Imaging Kit” (Invitrogen) according to the manufacturer's instructions. The NucBlue™ Live reagent labels the nuclei of all the cells, while NucGreen™ Dead only labels the nuclei of dead cells. After 20 minutes of labeling, the matrices are washed 3 times in PBS, and fluorescence is detected by confocal microscopy (LSM880, Zeiss). The total number of cells, and also the number of dead cells, are evaluated using automated analysis of the photos on Fiji (a macro developed by the Restore laboratory) (analysis of 5 photos per condition).

### Measurement of Cytokine Production by Human Macrophages and Human ASCs Seeded In the Matrix

Human ASCs (1×10⁵ cells) and different populations of human Mφ (1×10⁵ cells) are seeded alone or in combination in the 3D matrix. After 12 h of seeding, the medium is replaced and the matrices containing the cells are re-cultured and stimulated, or not stimulated, with LPS (10 ng/ml). After 24 h of culture, the supernatants are recovered and the concentrations of IL-6, IL-1β and IL-10 are determined by ELISA according to the supplier's instructions (Invitrogen).

### RESULTS

### A) Analysis of the Retention, Viability and Distribution of the Macrophages in the 3D Matrices

In order to validate the 3D matrices as supports for pro-resolving M2 macrophages for cell therapy, the retention, viability and distribution of these macrophages within the 3D matrices were studied. The level of retention of the mΦ M2 after seeding was evaluated by quantification of the total extracted proteins.

The results show that the level of retention is high (approximately 85%).

The study of the viability of the M2 macrophages within the PEC40/60 3D matrix was monitored qualitatively by “live imaging” confocal microscopy. The hydrated 3D matrices were imaged longitudinally and transversally (FIG. 8). Analysis of the reconstructions made it possible to demonstrate viability of the M2 macrophages of 89±3% after 24 hours of seeding in the 3D matrix.

This labeling also provides information about the spatial distribution of the mΦ M2 in the matrices. No regionalization is observed. On the contrary, the mΦ M2 are homogeneously distributed both at the surface and at depth.

Interestingly, after 21 days of culture, the mΦ M2 still have good viability in the 3D matrices.

In conclusion, the PEC 40/60 3D matrices combining alginate and chitosan constitute an optimal 3D support for M2 macrophages for cell therapy.

### B) Effect of the Matrix According to the Invention on the Phenotype of the Macrophages.

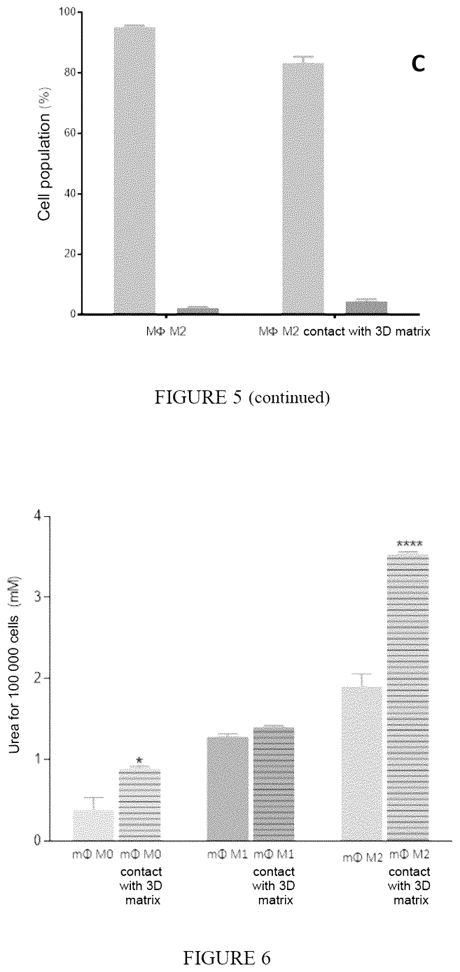

The effect of the alginate-chitosan 3D matrices on the polarization of macrophages activated toward a phenotype, M0, M1 and M2, was studied. To this end, alginate-chitosan (40/60) matrices were brought into contact with macrophages (mΦ M0, M1 or M2) for 24 hours and the expression level of mRNAs encoding pro- and anti-inflammatory cytokines (Tnf-α, Il-10, Tgf-β1) was studied (FIG. 4). The gene and protein expression of membrane receptors characteristic of the phenotypes M1 (Cd86) and M2 (Cd206, Cd36) was also studied (FIG. 4 and FIG. 5).

This initial phenotypic characterization was then supplemented with functional analyses, such as the study of arginine metabolism or the production of reactive oxygen species (ROS).

First of all, these analyses made it possible to validate the polarization M0, M1 or M2 of the macrophages in culture after isolation thereof from bone marrow and treatment with various inducers. Furthermore, they demonstrate that, in contact with the alginate-chitosan 3D matrices, the macrophages orient themselves toward an M2 phenotype. Indeed, the alginate-chitosan 3D matrices appear to potentiate the phenotype of M2 macrophages by increasing the expression of M2 markers (Il-10, Tgf-β1, Cd206, Cd36) and by decreasing the expression of M1 markers (Tnf-α, Cd86), and also to orient non-activated M0 macrophages toward an M2 phenotype. Just as interestingly, the M1 macrophages in contact with the matrices exhibit weaker expression of the pro-inflammatory markers characteristic of M1 macrophages (Tnf-α and Cd86).

Together, these results demonstrate that bringing the M2 macrophages into contact with the alginate-chitosan 3D matrices does not modify their phenotype. Moreover, the matrices appear to orient M0 and M1 macrophages toward an M2 phenotype and to potentiate the M2 phenotype of M2 macrophages.

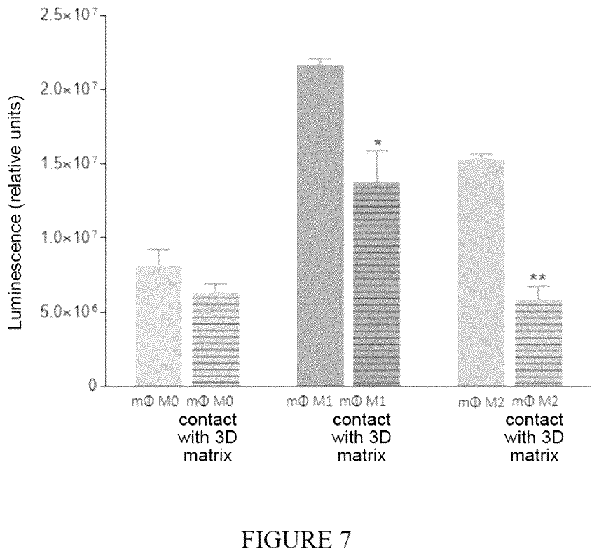

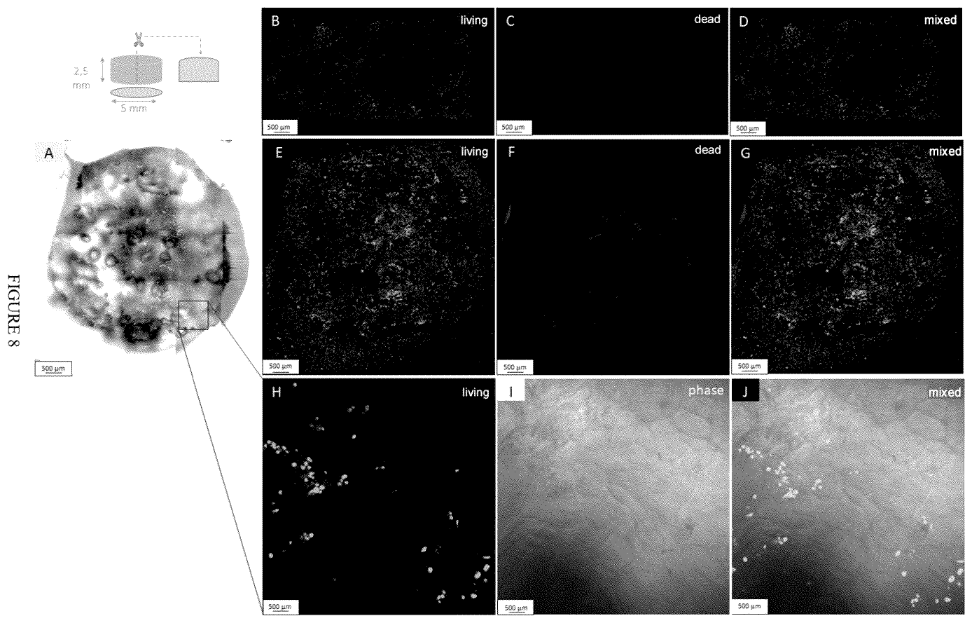

With the aim of exploring the impact of the alginate-chitosan matrices on the effector functions of the macrophages, arginine metabolism (FIG. 6) and oxidative stress (FIG. 7) were evaluated. Arginine metabolism is key to the various functions associated with the M1 and M2 phenotypes of macrophages (mΦ). Indeed, nitric oxide synthase (NOS) and arginase compete for the substrate L-arginine (Mills, 2012; Rath et al., 2014; Shearer et al., 1997). In particular, urea is often assayed to evaluate the activity of arginase, which is a characteristic enzyme of M2 macrophages (Csonka et al., 2015). M0 and M2 macrophages exhibit significantly greater urea production in contact with the alginate-chitosan 3D matrices, demonstrating greater arginase activity in these macrophages (FIG. 6).

Regarding ROS production, as expected, the ability of M1 macrophages to produce ROS is greater than that of M0 and M2 macrophages following TPA stimulation. This ability to produce ROS is greatly decreased in the M1 and M2 macrophages brought into contact with the matrices (FIG. 7). These results show that the alginate-chitosan 3D matrices are capable of orienting the functions of M0 and M1 macrophages toward functions characteristic of M2 macrophages, and of potentiating the specific functions of M2 macrophages.

In conclusion, together, these first results made it possible to demonstrate that the phenotype and functions of M2 macrophages are maintained by the 3D matrices according to the invention. Moreover, the 3D matrices appear to orient M0 macrophages toward an M2 phenotype and to attenuate M1 polarization.

### C) In Vivo Study of the Combination of Pro-Resolving Macrophages/3D Matrices on a Mouse Wound Model in a Context of Type 2 Diabetes, According to the First Protocol

Before the wounds are made, the fasting blood sugar and the weight of the mice on the HFD diet were verified. These values were compared to a “normal chow” control group consisting of C57Bl/6 mice fed normally. The average weight of the mice on the high-fat diet (HFD) is higher than that of the control mice (51.5±0.5 g vs 25.2±0.9 g). As for the fasting blood sugar, it reached a significantly higher value than in the control group (183.2±6.3 mg/dL vs 128.3±4.5 mg/dL). Once this model was validated, skin wounds of approximately 1 cm2 were made on the dorsal part by circular excision. Immediately thereafter, a non-invasive experimental device (FR2984719/FR2984722) was implanted. This makes it possible, inter alia, to keep the 3D matrices on the wounds. The treatments started 48 hours after the wounds were made, so as not to interfere with the inflammatory phase, the correct functioning of which enables the activation of the subsequent wound healing phases (Jetten et al., 2014).

The treatments consist in topically applying, to the wound, culture medium (control group), 200 000 M2 macrophages (mΦ M2), or a hydrated 3D matrix alone or seeded with 200 000 M2 macrophages.

By virtue of the non-invasive experimental device, wound closure was monitored daily by taking standardized photographs from the day on which the wound was made, DO, to the day the mice were sacrificed, D15.

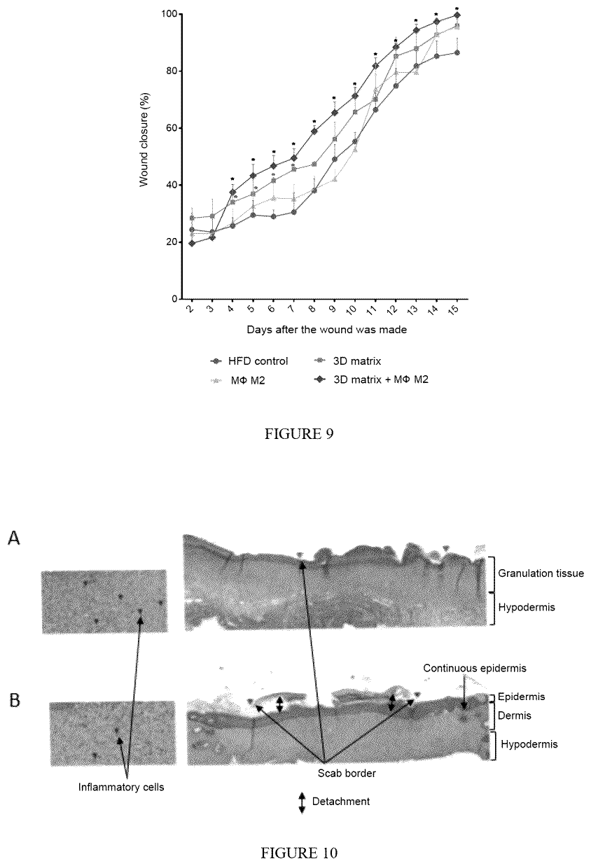

From a quantitative perspective, wound closure was determined by daily measurement of the surface area of the wounds for each mouse. The results are presented as percentage wound closure relative to the surface area of said wounds on the day on which treatment began (D2) (FIG. 9).

It is thus possible to observe that the local addition of M2 macrophages to the wounds does not improve wound closure. Interestingly, the application of the hydrated 3D matrices alone accelerated wound closure, with a more significant effect at the earlier stages (D4 to D7). This result is in correlation with the orientation of the M0 macrophages toward an M2 phenotype and with the attenuation of the M1 profile, observed when the macrophages were brought into contact with the 3D matrices. From D4 to D15, the application of the 3D matrices seeded with M2 macrophages significantly accelerates wound closure compared to the control group.

Thus, the beneficial pro-wound-healing effect of the matrices alone is potentiated by the addition of M2 macrophages. These results show that the combination of the PEC 40/60 matrices with pro-resolving macrophages enables more rapid wound closure. The combination of the matrix according to the invention with pro-resolving macrophages exhibits a synergistic effect on wound closure.

The biomaterial according to the invention therefore makes it possible to (i) act as a barrier to infectious agents in the environment, (ii) when in contact with the wound, orient endogenous macrophages in the wound toward an anti-inflammatory phenotype, but also (iii) maintain the viability and pro-resolving functionality of the macrophages.

### D) Evaluation of Wound-Healing Quality

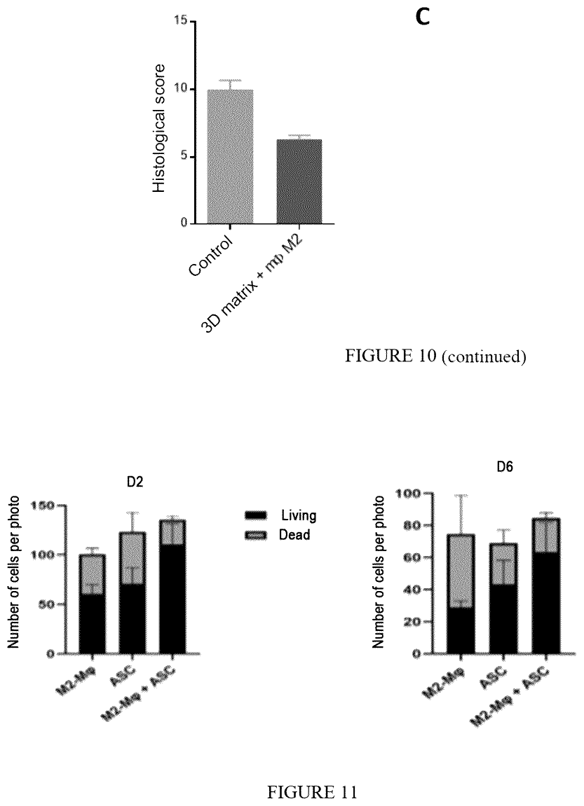

In order to investigate the quality of the newly-formed skin, hematoxylin and eosin staining was carried out on histological sections produced on D15. Several scores were developed in order to standardize the qualitative analysis of the skin sections after wound healing (Gantwerker & Hom, 2011; Gupta & Kumar, 2015) (FIG. 10).

The score was determined on the basis of the following parameters: the presence of inflammatory cells; the quality of the epidermis (continuity and thickness relative to healthy skin); the quality of the epidermis (continuity, presence of hair follicles); the presence of a scab (if so, detachment and/or presence of inflammatory cells).

The slices were qualitatively analyzed by two different operators, and scores ranging from 0 to 15 were assigned (the score is lower when the quality of the newly-formed skin is high). The histological score is lower in mice that were treated with the 3D matrix seeded with the pro-resolving macrophages.

The histological analysis of the skin sections by hematoxylin and eosin staining revealed a better scar tissue quality in terms of continuity of the dermis and epidermis, and also a lower presence of inflammatory cells at the wounds treated by the 3D matrices combined with pro-resolving macrophages.

### E) Viability and Functionality of the Various Cell Types (Human M2-Mφ and Human ASCs) Combined in the Matrix

The results show that, although the macrophages and the ASCs were seeded in identical amounts, from D2 the ASCs are present in a greater number, thus revealing slightly higher viability than the M2-MΦ. Very interestingly, the combination of the two cell types significantly increases the number of both cell types, and also their viability in the matrix (FIG. 11).

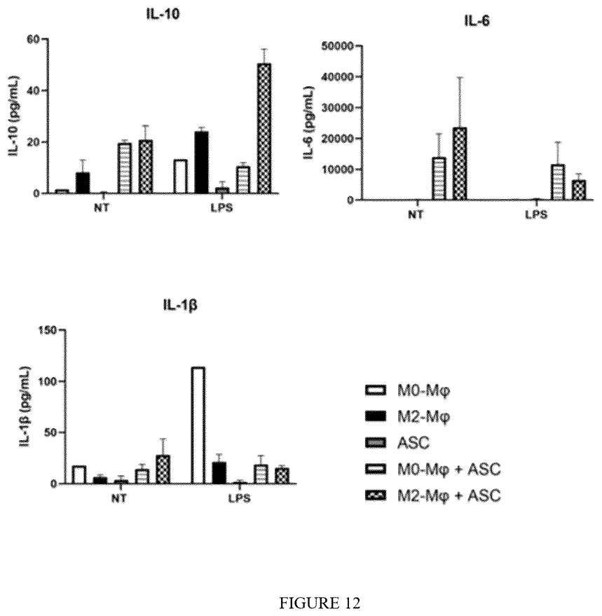

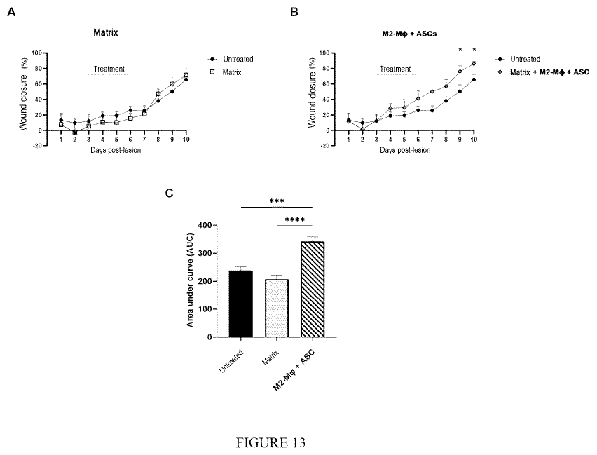

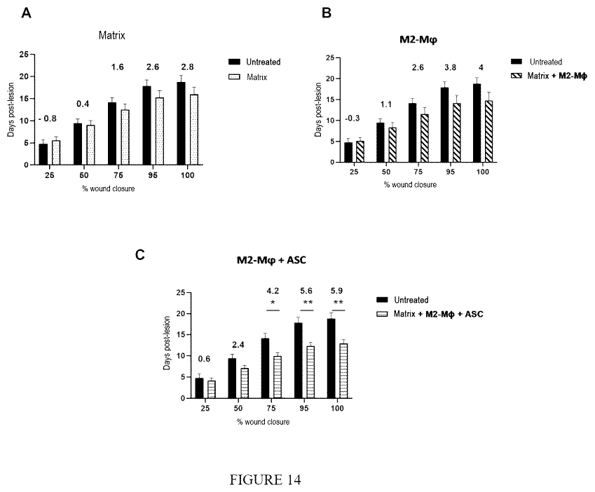

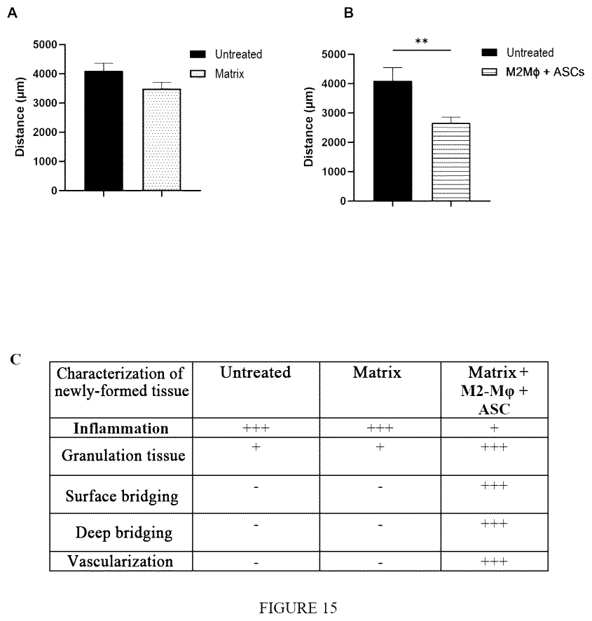

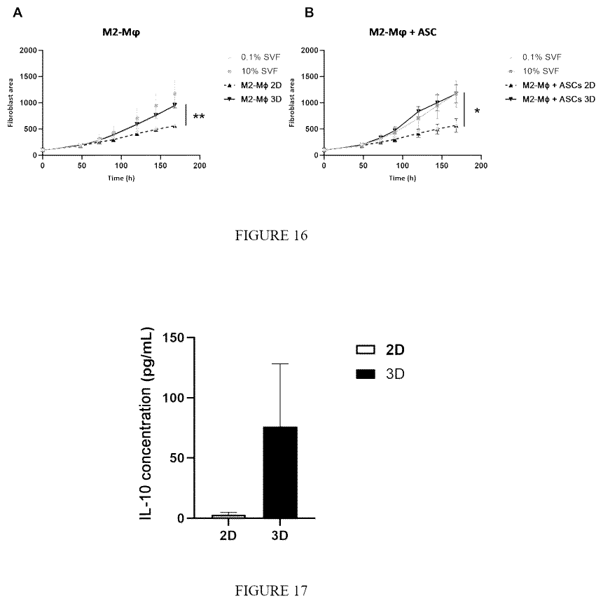

Very interestingly, the results show that the combination of the ASCs and the various types of macrophages induces greater production of IL-10 and an explosive production of IL-6, two cytokines which have a central role in inflammation resolution and tissue repair. In the presence of LPS, the induction of IL-1 beta production by the macrophages in the matrix is strongly inhibited by the co-seeding with the ASCs (FIG. 12).

Together, these results demonstrate the benefit of combining macrophages and ASCs in the matrix to increase the viability thereof and to promote a pro-resolving and repairing environment.

### F) In Vivo Study of the Combination of Pro-Resolving Macrophages/ASCs in the 3D Matrix on a Mouse Wound Model in a Context of Type 2 Diabetes

Following the results obtained previously (C-E), a second in vivo mouse protocol was carried out in order to evaluate the effect of the combination of M2 macrophages with ASCs in the matrix on wound healing in a context of type 2 diabetes. By virtue of the non-invasive experimental device used previously, wound closure was monitored daily by taking standardized photographs from the day on which the wound was made, DO, to the day the mice were sacrificed, D14.

From a quantitative perspective, wound closure was determined by daily measurement of the surface area of the wounds for each mouse. The results are presented as percentage wound closure on D14 (sacrifice) relative to the surface area of said wounds on the day on which the wound was made. As expected (FIG. 9), the application of the 3D matrices seeded with M2 macrophages improves wound closure compared to the control group. Interestingly, the combination of the M2 macrophages with the ASCs significantly increases the percentage wound closure on D14 compared to the mice that received no treatment.

Moreover, quantification of the filling tissue clearly shows more filling tissue in the group that received the matrix containing M2 macrophages and ASCs compared to the other groups.

Thus, the beneficial pro-wound-healing effect of the matrices seeded with M2 macrophages appears to be potentiated by adding ASCs. These results show that the combination of the PEC 40/60 matrices with pro-resolving macrophages and ASCs enables the wounds to be filled and more rapid wound closure.

1-25. (canceled)

26. A three-dimensional hybrid biomaterial comprising a matrix of polyelectrolyte complexes (PEC) based on modified or unmodified alginate and chitosan, and macrophages, said matrix having an open, interconnected macroporosity in which the alginate/chitosan weight ratio is between 20/80 and 80/20.

27. The hybrid biomaterial as claimed in claim 26, wherein the alginate/chitosan weight ratio is 40/60.

28. The hybrid biomaterial as claimed in claim 26, wherein the alginate has at least one property selected from an M/G ratio of between 1.4 and 2.7, a molecular weight (Mw) of between 150,000 and 250,000 and a polydispersity index (PI) of less than 2.

29. The hybrid biomaterial as claimed in claim 26, wherein the chitosan has at least one property selected from a degree of deacetylation (DDA) of between 75% and 90%, an Mw of between 130,000 and 400,000, and a polydispersity index of less than 2.

30. The hybrid biomaterial as claimed in claim 26, comprising pro-wound-healing macrophages.

31. The hybrid biomaterial as claimed in claim 30, wherein the pro-wound-healing macrophages are pro-resolving macrophages.

32. The hybrid biomaterial as claimed in claim 26, comprising pro-wound-healing macrophages and mesenchymal stromal cells (MSCs), the pro-wound-healing macrophages/mesenchymal stromal cells ratio being between 1/99 and 99/1.

33. The hybrid biomaterial as claimed in claim 26, wherein the alginate/chitosan weight ratio is 40/60, the alginate has at least one property selected from an M/G ratio of between 1.4 and 2.7, a molecular weight (Mw) of between 150,000 and 250,000 and a polydispersity index (PI) of less than 2 and the chitosan has at least one property selected from a degree of deacetylation (DDA) of between 75% and 90%, an Mw of between 130,000 and 400,000, and a polydispersity index of less than 2.

34. The hybrid biomaterial as claimed in claim 26, wherein the alginate/chitosan weight ratio is 40/60, the alginate has an M/G ratio equal to 2+/−0.2, a molecular weight (Mw) of between 150,000 and 250,000, and a polydispersity index (PI) of approximately 1.5, and the chitosan has a degree of deacetylation (DDA) of between 75% and 85%, an Mw of between 145,000 and 400,000, and a polydispersity index of approximately 1.8.

35. A method for preparing a hybrid biomaterial, comprising the steps of:
(a) culturing monocytes and/or macrophages under conditions that produce pro-wound-healing macrophages;
(b) preparing a matrix of polyelectrolyte complexes (PEC) based on alginate and chitosan in relative proportions of between 40/60 and 60/40;
(c) optionally drying the PEC matrix;
(d) sterilizing the PEC matrix;
(e) seeding the PEC matrix with pro-wound-healing macrophages obtained in step (a), optionally combined with MSCs.

36. The method for preparing a hybrid biomaterial as claimed in claim 35, wherein sterilization step (d) is a sterilization by low-energy pulsed electron beam irradiation or a sterilization by low-energy continuous electron beam irradiation.

37. A cutaneous wound dressing intended for regenerative medicine, comprising a three-dimensional hybrid biomaterial as claimed in claim 26.

38. A kit for regenerative medicine, said kit comprising:
an alginate and chitosan PEC matrix, said matrix having an open, interconnected macroporosity in which the anionic polymer/cationic polymer weight ratio is between 20/80 and 80/20;
at least one culture medium selected from a culture medium suitable for culturing monocytes or pro-wound-healing macrophages, and a culture medium suitable for differentiating bone marrow cells into macrophages of a pro-wound-healing phenotype.

39. The kit as claimed in claim 38, wherein the PEC matrix is sterilized.

40. A method for treating damaged tissue comprising applying a hybrid biomaterial as claimed in claim 26 to damaged tissue to promote the repair of said tissue.

41. The treatment method as claimed in claim 40, according to which said hybrid biomaterial contains pro-wound-healing macrophages and, optionally, MSCs.

42. The treatment method as claimed in claim 40, according to which said hybrid biomaterial contains pro-resolving macrophage.

43. The treatment method as claimed in claim 42, according to which the hybrid biomaterial also comprises MSCs.

44. A cutaneous wound dressing intended for regenerative medicine, comprising a three-dimensional hybrid biomaterial as claimed in claim 34.

45. A method for treating damaged tissue, according to which a hybrid biomaterial as claimed in claim 34 is applied to the damaged tissue in order to promote the repair of said tissue.


SEQUENCE LISTING (12 sequences)

SEQ ID NO: 1 - DNA, 20 bp, synthetic construct
  source 1..20: mol_type = other DNA
aactttggcattgtggaagg

SEQ ID NO: 2 - DNA, 20 bp, synthetic construct
  source 1..20: mol_type = other DNA
acacattgggggtaggaaca

SEQ ID NO: 3 - DNA, 20 bp, synthetic construct
  source 1..20: mol_type = other DNA
ttttcacaggggagaaatcg

SEQ ID NO: 4 - DNA, 20 bp, synthetic construct
  source 1..20: mol_type = other DNA
ccaagccttatcggaaatga

SEQ ID NO: 5 - DNA, 20 bp, synthetic construct
  source 1..20: mol_type = other DNA
aggttggcattccacttcac

SEQ ID NO: 6 - DNA, 20 bp, synthetic construct
  source 1..20: mol_type = other DNA
aggggcctctaagagcagtc

SEQ ID NO: 7 - DNA, 20 bp, synthetic construct
  source 1..20: mol_type = other DNA
aggctgtgcattgcacctca

SEQ ID NO: 8 - DNA, 20 bp, synthetic construct
  source 1..20: mol_type = other DNA
gggacagtgacctgcactgt

SEQ ID NO: 9 - DNA, 20 bp, synthetic construct
  source 1..20: mol_type = other DNA
atgccaagtgggaaaatctg

SEQ ID NO: 10 - DNA, 20 bp, synthetic construct
  source 1..20: mol_type = other DNA
tgtagcagtggcctgcatag

SEQ ID NO: 11 - DNA, 20 bp, synthetic construct
  source 1..20: mol_type = other DNA
gcagaatcaagggagagcac

SEQ ID NO: 12 - DNA, 20 bp, synthetic construct
  source 1..20: mol_type = other DNA
gagcaactggtggatggttt